(19) United States
(12) Patent Application Publication
(10) Pub. No.: US 2024/0130646 A1
(43) Pub. Date: Apr. 25, 2024

(54) NON-INVASIVE AND NON-CONTACT BLOOD GLUCOSE MONITORING WITH HYPERSPECTRAL IMAGING

(71) Applicant: Algorithmic Mashup, Inc., La Jolla, CA (US)

(72) Inventors: Alan Blair Thomas, Gig Harbor, WA (US); Harsh Sunilkumar Thaker, Czechowice-Dziedzice (PL)

(21) Appl. No.: 18/485,590

(22) Filed: Oct. 12, 2023

Related U.S. Application Data
(60) Provisional application No. 63/379,693, filed on Oct. 14, 2022.

(51) Int. Cl. A61B 5/145; A61B 5/00; G06V 10/774; G06V 10/82

(52) U.S. Cl. CPC A61B 5/14532; A61B 5/6898; A61B 5/7267; G06V 10/774; G06V 10/82; G06V 10/764; G06V 2201/03

(57) ABSTRACT

A method and system for deriving a blood glucose level of a user is disclosed. One or more images of the user are captured with a hyperspectral imaging device, and the images may be defined by a plurality of layered data sets, each of which correspond to an electromagnetic spectrum band channel. The one or more images of the user are fed to a machine learning model that is trained on a plurality of correlated pairs of one or more training images associated with training blood glucose measurements. An estimated blood glucose level corresponding to the one or more images of the user is generated with the machine learning model.

### CROSS-REFERENCE TO RELATED APPLICATIONS

This application relates to and claims the benefit of U.S. Provisional Application No. 63/379,693 filed Oct. 14, 2022 and entitled “NON-INVASIVE AND NON-CONTACT BLOOD GLUCOSE MONITORING WITH HYPERSPECTRAL IMAGING,” the entire disclosure of which is wholly incorporated by reference herein.

### STATEMENT RE: FEDERALLY SPONSORED RESEARCH/DEVELOPMENT

Not Applicable

### BACKGROUND

### 1. Technical Field

The present disclosure relates generally to imaging systems and blood glucose monitoring, and more particularly to non-invasive and non-contact blood glucose monitoring with hyperspectral/multispectral imaging and machine learning.

### 2. Related Art

Diabetes mellitus, or more simply diabetes, is an incurable chronic condition in which blood glucose levels cannot be properly regulated as a consequence of the pancreas being unable to produce sufficient insulin, or the body being unable to utilize insulin. In some cases, the production of insulin may be stopped altogether because of an autoimmune reaction, with this form being referred to as type 1 diabetes. With type 2 diabetes on the other hand, insulin is still being produced, but elevated blood sugar levels are sustained because the cells of the body have developed insulin resistance and the ability for such cells to absorb glucose has diminished. Approximately 5 to 10 percent of diabetes patients are estimated to be afflicted with type 1, while type 2 accounts for over 90 to 95% of the patient population. Chronically elevated blood sugar levels are understood to result in several major health complications, including cardiovascular diseases such as coronary artery disease, heart attacks, strokes, and so forth, along with kidney damage, eye damage, limb damage, and so on.

In the United States, the Centers for Disease Control estimates that more than 37 million adults have diabetes and is the seventh leading cause of death. Worldwide, there are estimated to be over 463 million adults with diabetes. Furthermore, over 96 million or 38% of U.S. adults are estimated to have pre-diabetes, where there is a reduced capacity to produce and/or process insulin and results in higher than normal blood sugar levels but are not as elevated as the baseline for a diabetes diagnosis.

Although type 1 diabetes is oftentimes accompanied by symptoms such as extreme fatigue, excessive thirst and hunger, frequent urination, blurred vision, and numb/tingling extremities, type 2 diabetes may go unnoticed for much longer. Indeed, it is estimated that over 8.5 million U.S. adults who currently have diabetes may be undiagnosed, and over 80% of those with pre-diabetes may be undiagnosed. Accordingly, early detection, monitoring, and intervention for diabetes are critical. Once diagnosed, managing diabetes may involve a combination of lifestyle changes such as healthier diets, losing weight, and more exercise, insulin intake by various routes, or other medication.

No matter the severity, the cornerstone of diabetes management is the regular monitoring of blood glucose levels. By maintaining ideal or close to ideal blood glucose levels, the more severe complications may be avoided, and feedback from the lifestyle changes may be immediately available. Conventionally, blood glucose monitoring is an uncomfortable process that requires the patient to draw blood in some fashion—typically via a lancet that pricks the finger. The blood sample may be deposited on a test strip that is read by a glucometer to derive a blood glucose value. Type 1 diabetes patients may need to test multiple times throughout the day to determine insulin dosages, while the frequency may be substantially reduced. Because of the high costs associated with test strips as well as the pain and sanitation/wound care requirements associated with drawing blood, there has been a long-standing demand for non-invasive glucose monitoring.

A variety of non-invasive glucose monitoring technologies have been attempted, though none successfully as of yet. These approaches include mid-infrared spectroscopy and near-infrared spectroscopy in which images of a specific body part captured by sensors are evaluated for correspondence to specific blood glucose levels. Additionally, microwave/radio frequency-based sensors, along with ultrasound, bioimpedance, fluorescence, and Raman spectroscopy technologies have been attempted. Others have attempted to electrically, ultrasonically, or chemically sense glucose levels through transdermal measurements. However, many of these approaches require extensive laboratory equipment, and reliability and accuracy were less than desirable.

More recently, there have been efforts toward utilizing smartphones for blood glucose monitoring because of their ubiquity in everyday life. One involves photoplethysmography (PPG), or the volumetric change of blood in the arteries, derived from video or sequences of images captured by on-board cameras and making evaluations with neural networks and other machine learning techniques correlating the derived features to specific blood glucose levels. Such developments are disclosed in U.S. Pat. App. Pub. No. 2022/0117524 to Yeh et al. The aforementioned near-infrared imaging technique is disclosed in U.S. Pat. App. Pub. No. 2022/0079477 to Deng.

In order for non-invasive blood glucose measurement techniques to see widespread acceptance, the accuracy and reliability would need to reach at least the same level as conventional blood sampling glucometers. The mean absolute relative difference would thus need to be below 20%. Accordingly, there is a need in the art for an improved non-invasive and non-contact blood glucose monitoring system with improved accuracy and reliability.

### BRIEF SUMMARY

According to an embodiment of the present disclosure, there may be a method for deriving a blood glucose level of a user. The method may include capturing one or more images of the user with a hyperspectral imaging device. The images may be defined by a plurality of layered data sets, each of which may correspond to an electromagnetic spectrum band channel. The method may also include feeding the one or more images of the user to a machine learning model trained on a plurality of correlated pairs of one or more training images associated with training blood glucose measurements. There may also be a step of generating, with the machine learning model, an estimated blood glucose level for the user corresponding to the one or more images thereof.

The method may further include capturing the one or more training images of a plurality of training users with the hyperspectral imaging device. The training images may be defined by a plurality of layered data sets each corresponding an electromagnetic spectrum band channel. There may also be a step of capturing the training blood glucose measurements of the training users concurrently with the capturing of the training images. The method may also include feeding one or more correlated pair of the training blood glucose measurement and the training images to the machine learning model.

According to another embodiment, there may also be a non-transitory program storage medium on which are stored instructions executable by a processor or programmable circuit to perform the foregoing method for deriving a blood glucose level of a user.

Another embodiment of the present disclosure may be an apparatus for monitoring a blood glucose level of a user. The apparatus may include a hyperspectral imaging device. One or more images of the user may be captured by the hyperspectral imaging device with each being defined by a plurality of layered data sets each corresponding to an electromagnetic spectrum band channel. The apparatus may also include a glucose level evaluation interface that is in communication with a machine learning model trained on a plurality of correlated pairs of one or more training images and associated training blood glucose measurements. The one or more images of the user from the hyperspectral imaging device may be relayed to the machine learning model. The glucose level evaluation interface may also be receptive to an estimated blood glucose level generated in response to the one or more images.

The present disclosure will be best understood accompanying by reference to the following detailed description when read in conjunction with the drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a block diagram of one exemplary embodiment of a blood glucose level monitoring system;

FIG. 2 is an example representation of a conventional imaging sensor output;

FIG. 3 is an example representation of a hyperspectral imaging sensor output;

FIG. 4 is a detailed block diagram of the blood glucose level monitoring system including its constituent functional components;

FIG. 5A is a block diagram illustrating a regression model machine learning for the blood glucose level monitoring system;

FIG. 5B is a block diagram illustrating a classification model machine learning for the blood glucose level monitoring system; and

FIG. 5C is a block diagram illustrating a multi-task model machine learning combining the regression model and the classification model for the blood glucose level monitoring system.

### DETAILED DESCRIPTION

The detailed description set forth below in connection with the appended drawings is intended as a description of the several presently contemplated embodiments of methods and apparatus for monitoring blood glucose levels and is not intended to represent the only form in which such embodiments may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

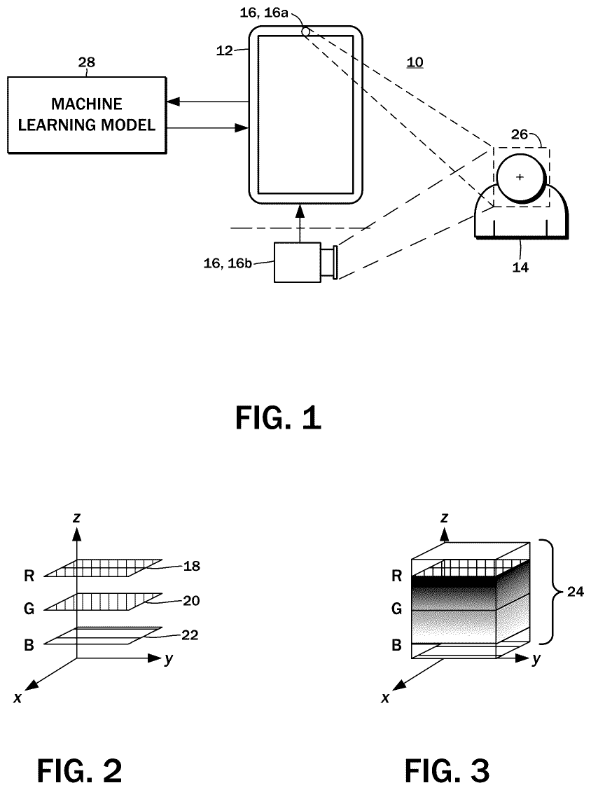

The embodiments of the present disclosure contemplate the non-invasive and non-contact monitoring of blood glucose levels. With reference to FIG. 1, the blood glucose monitoring system 10 may be incorporated into a smartphone 12 or other portable electronic device that is expected to be carried by a user 14 while going about everyday activities. Although the embodiments of the system 10 will be disclosed in the context of such smartphone 12, it will be appreciated by those having ordinary skill in the art that other devices such as tablets, laptop computers, or dedicated blood glucose monitoring devices that incorporate the contemplated features of the system 10 may be substituted.

The non-invasive and non-contact blood glucose monitoring is envisioned to be possible based upon an analysis of image data captured of the user 14. In this regard, the smartphone 12, and the blood glucose monitoring system 10, may include or be connected to a camera/imaging device 16. FIG. 2 illustrates an exemplary output of a conventional imaging sensor that is sensitive only to the three primary colors of red, green, and blue, with a single array representative of the image field being generated for each primary color sensitivity. Specifically, there is a red-band array 18, a green-band array 20, and a blue-band array 22. A typical sensor array is fabricated on a single plane and comprised of photodetectors. A first subset of photodetectors may be located behind a red colored filter, a second subset of photodetectors may be located behind a green colored filter, and a third subset of photodetectors may be located behind a blue colored filter, with the various color filters being arranged in grouped patterns. This configuration is known as the Bayer-filter sensor, though other configurations for separating different color wavelengths before reaching the monochromatic photodetector are known in the art. The spatial resolution of a given sensor is understood to refer to the number of individual pixels in the sensor field.

FIG. 3 illustrates an exemplary output of a hyperspectral imaging (HSI) device. Although the conventional sensor only detects the discrete narrow bands of the visible portion of the electromagnetic spectrum, the hyperspectral imaging device is capable of detecting a continuous and contiguous range 24 of wavelengths extending beyond the visible electromagnetic spectrum. In one implementation, the sensitivity of the HSI sensors may be between approximately 10 nanometers to approximately 0.1 millimeters, in 1 nanometer steps. Other embodiments of the disclosure may utilize what is referred to as multi-spectral imaging, where the gradations between each of the steps in either the same or different range (e.g., approximately 400 nm to approximately 1100 nm) is increased to around 20 nm. The spectral resolution, or the width of each band of the spectrum captured by the sensor, is decreased. However, there may be improved processing efficiencies as a consequence of a smaller data set. It will be appreciated that the specific sensitivity range of the HSI sensor is presented by way of example only and not of limitation, and other imaging devices may have different sensitivity ranges.

The embodiments of the present disclosure are contemplated to leverage the additional information contained in each of the hyperspectral images in the contiguous range 24 as potential unique fingerprints or spectral signatures to make evaluations about a state of the user 14, e.g., the blood-glucose level. As a general matter, the spectral signatures captured by the HSI imaging devices may enable the identification of the materials that comprise the object being scanned or captured.

The specific configuration of the HSI device may vary, though the balance between spatial and spectral resolution may be optimized from application to application depending on the needed detection speeds and sensitivity requirements for identifying the spectral signatures of interest. The identification of objects may still be possible by capturing a large number of relatively narrow frequency bands. If the pixel size is too large, multiple objects or discrete elements of the spectral signatures may be captured and become difficult to individually identify. On the other hand, if the pixel size is too small, the intensity of the electromagnetic radiation captured by a given sensor cell may be too low, thereby decreasing the signal-to-noise ratio and degrading the reliability of measured features. Hyperspectral imaging is understood to find application in facial recognition systems for authenticating users to an access-restricted service. According to various embodiments of the present disclosure, hyperspectral imaging is extended to blood-glucose level evaluations.

Referring again to the block diagram of FIG. 1, the smartphone 12 may have an integrated imaging device 16a. In some implementations of the smartphone 12, the imaging device may be the aforementioned hyperspectral imaging sensor capable of capturing the continuous range of electromagnetic radiation within the visual spectrum as well as those parts of the spectrum beyond the visible portion, e.g., ultraviolet, infrared, and so forth. In some cases, the integrated imaging device 16a may be a conventional visible spectrum sensor, in which case the smartphone 12 may be connected to an external imaging device 16b that includes an HSI sensor. A variety of modalities may be employed to so connect the external imaging device 16b to the smartphone 12, including wired connections such as USB, as well as wireless connections such as WiFi and Bluetooth. As referenced herein, the imaging device 16 is understood to encompass the hyperspectral imaging sensor, along with any optical components needed for focusing onto the imaging sensor, as well as amplifiers and digital image processing circuitry that generates the final hyperspectral image data.

Regardless of the form factor, the imaging device 16 captures a scene 26, which may be a portion of the user 14. In a preferred, though optional embodiment, the portion of the user 14 that is captured for blood-glucose level evaluation is the face, though other body parts may be substituted, such as the wrists, the arms, and others. Although the face may often include various accoutrements such as glasses, scarfs, or partially covered by hair, the user 14 may be instructed to remove these obstructions while the scene 26 is captured. Along these lines, although the smartphone 12 may selectively focus the imaging device 16 onto the face, there may be extraneous objects in the background, or the background scene itself may contain irregular patterns that may make an analysis of the pertinent segments of the user 14 difficult. The smartphone 12 may perform various pre-processing steps that identify such extraneous objects and instruct the user 14 to remove them from the scene 26. Furthermore, consistent lighting conditions are also preferable, so further instructions along these lines may be presented during the capture process. Various sensors embedded into the smartphone 12 may be utilized to report ambient light condition so that the user 14 may add or subtract scene illumination to achieve pre-determined ideal levels.

The HSI capture process may be hindered by excessive movement of the smartphone 12 or the external imaging device 16b. Similar to the above-described pre-processing steps to identify and direct the removal of extraneous objects from the scene 26, the smartphone 12 may be configured to identify movement or shaky images through known pre-processing steps. Upon detection of such flaws in the capture process, the user 14 may be directed to re-capture the scene 26.

For avoiding the aforementioned issues with the capturing of a single image/hyperspectral image, a sequence of images, that is, a short video of the same scene 26 may be captured by the imaging device 16. In such cases, there may be pre-processor that evaluates each individual image in the sequence constituting the video against various quality metrics and select the best one(s) for further processing.

In addition to the pre-processing steps described above, the quality of the hyperspectral image may be further improved with a face detection process. Although facial recognition with conventional RGB images is well known and tailored specifically for such trichromatic data, hyperspectral image data may present additional challenges due to the richness of spectral information contained therein. To bridge the gap while leveraging established facial detection techniques, the hyperspectral images may be transformed into an RGB format. The objective of this conversion is to retain the most discriminative features across the hyperspectral bands to ensure reliable face detection while working within the constraints of the RGB-based procedures. According to one embodiment of the present disclosure, a YOLOv8 (You Only Look Once version 8) face detector may be used to identify and extract bounding boxes around detected faces. Utilizing the coordinates of the bounding box, the corresponding regions of the face in the hyperspectral images may be cropped out. Thus, existing RGB-based facial detection techniques may be employed while retaining the hyperspectral data for subsequent analysis.

After successful facial recognition and the hyperspectral images are cropped, the embodiments of the present disclosure contemplate the efficient storage of the extracted facial data. In one implementation, the data may be serialized, with the cropped facial regions being stored as binary files. An exemplary embodiment of the system may utilize a Python environment, and the binary data may be stored using the .npy format that is native to the NumPy library. It will be appreciated that binary storage facilitates rapid input/output operations. The selected binary storage format may retain array structure and data type information, thereby ensuring that no critical metadata is lost during the storage process. Furthermore, the format is widely used in scientific and machine learning applications, so sharing of the stored data is possible without complex conversions being necessary. As a general matter, this binary storage format is contemplated to ensure that facial data extracted from the hyperspectral images remain readily accessible while retaining its original integrity, and avoid unnecessary overhead and data loss.

The hyperspectral image data may also be normalized for each channel. It will be understood by those skilled in the art that hyperspectral images are comprised of multiple bands or channels, with each such band or channel capturing information at a specific wavelength. Because of the variability of spectral reflectance across different bands/channels, a normalization procedure may be applied to ensure consistent data scales that result in enhanced downstream processing effectiveness. According to one embodiment of the present disclosure, a channel-wide minimum-maximum normalization is applied to the hyperspectral image data. This is contemplated to account for the aforementioned variability, and harmonize the scale across different bands/channels. The normalization process involves scaling the data of each band/channel independently such that the minimum and maximum values map to a predefined range, for example, [0,1]. The normalizing transformation is defined as:

$${scaled}_{i , j} = \frac{( {pixel}_{i , j} - {min}_{j} )}{( {max}_{j} - {min}_{j} )}$$

Where scaled_{i,j} is the normalized value of the pixel i in the channel j, pixel_{i,j} is the original intensity value of the pixel i in the channel j, minⱼ is the minimum intensity value in the channel j, and minᵢ is the maximum intensity value in the channel j. The normalization process is contemplated to bound the data for each channel to a uniform range, thereby mitigating the potential for certain channels to disproportionately influence analytical outcomes stemming from scaling differences. The independent normalization of each channel is envisioned to preserve the relative variations within each channel, while achieving a consistent scale across the entirety of the hyperspectral image data set.

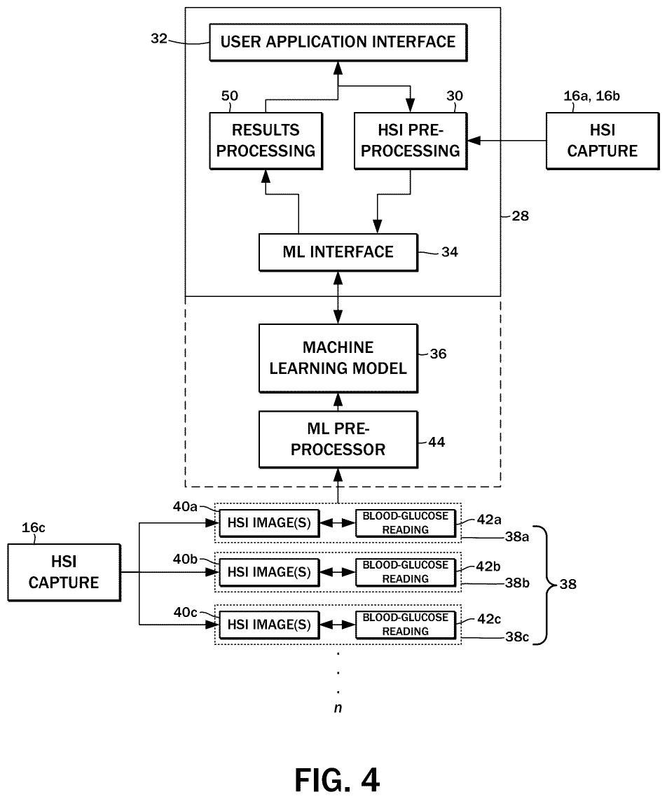

Again, the blood glucose monitoring system 10, or at least portions thereof, may be implemented on the smartphone 12 or other like apparatus that includes a general-purpose data processor capable of executing software instructions implementing the various functional features of the system 10. With additional reference to FIG. 4, the system 10 may be implemented as a blood glucose level monitoring application 28 comprised of specific components. As indicated above, the system 10, and hence the application 28, interfaces with a hyperspectral imaging device 16, the output of the device being provided to an HSI pre-processor 30. The captured scene 26 may be filtered, corrected, enhanced, or otherwise modified by the HSI pre-processor 30. Furthermore, to the extent problems in the captured scene 26 are detected by the HSI pre-processor 30 and it becomes necessary for the user 14 to take further corrective steps as discussed above, such instructions may be output by a user application interface 32.

The optimal hyperspectral images are then provided to a machine learning interface 34 that may be executing on the smartphone 12. The underlying data of the hyperspectral image may be provided to a machine learning model 36, which makes an evaluation of the blood glucose level of the user 14 from the provided hyperspectral image thereof.

This evaluation and the resultant conclusion is possible because the machine learning model 36 is trained to discern certain spectral signatures contained within the hyperspectral image as being correlated to certain blood glucose levels. This training is understood to be achieved with a training data set 38 from captured hyperspectral images 40 of a training user. Similar to the HSI captures of the user 14, there may be another HSI device 16c that captures hyperspectral images 40 of the training user at different times and under different blood-glucose conditions. Each of the training hyperspectral images 40 are paired with corresponding blood-glucose readings 42 taken concurrently, or at least substantially contemporaneously as the training hyperspectral image 40. To this end, there is also a first hyperspectral image 40a, a second hyperspectral image 40b, and a third hyperspectral image 40c. The blood-glucose readings 42 are understood to be conventionally taken via invasive blood glucose meters (BGM/CGM devices) discussed above, and provided in mg/dL. Any other medically approved blood glucose measurement apparatus may be substituted.

The first hyperspectral image 40a is linked with the first blood-glucose reading 42a to define a first training data pair 38a, the second hyperspectral image 40b is linked with the second blood-glucose reading 42b to define a second training data pair 38b, and the third hyperspectral image 40c is linked with the third blood-glucose reading 42c to define a third training data pair 38c. Each of the training data sets 38 may be readings taken throughout the day: upon waking, before eating, after eating, and so on. Furthermore, although the example of FIG. 4 only illustrates three training data pairs 38a-38c, there may be substantially more training data pairs for a given user spanning multiple times and dates. Additionally, the overall training data set 38 can be expanded to encompass training users of different racial backgrounds, skin types, gender, age, and other physical and demographic characteristics.

Before being input to the machine learning model 36, the training hyperspectral images 40 may be further optimized to eliminate extraneous information by a training pre-processor 44. Specifically, the training hyperspectral images 40 may be cropped and/or segmented to limit the captured scene to that of the body part of interest, e.g., the face. In one exemplary embodiment, a facial detection process may be used to identify the face within the image. The training hyperspectral image 40 may then be cropped to limits of the detected face. Like the cropping of the hyperspectral image taken of the user 14, cropping the training hyperspectral images 40 is understood to improve the training accuracy and thus achieve better results. Alternatively, or in addition, an image segmenting process may be applied to the captured training scene on a pixel- by-pixel basis so that only those portions corresponding to the face remain.

The training pre-processor 44 may pre-process the target or training hyperspectral images 40 for optimizing the machine learning model 36. The target dataset is understood to be comprised of image data, in which each of the images is associated with a target value that corresponds to a glucose score. In one embodiment, the target value/glucose score may range from 70 to 500, though this may vary. In order to improve the efficiency of the learning process and to improve the performance of the machine learning model 36, one possible implementation may incorporate a minimum-maximum scaling to normalizing the target values. The transformation is defined as:

$$scaled = \frac{( x - min )}{( max - min )}$$

Where x is the original target value, the min is the minimum target value in the dataset, and the max is the maximum target value in the dataset. After this transformation, all target values are understood to be scaled to fall within the range of [0, 1]. The training data sets 38 may then be provided to the machine learning model 36.

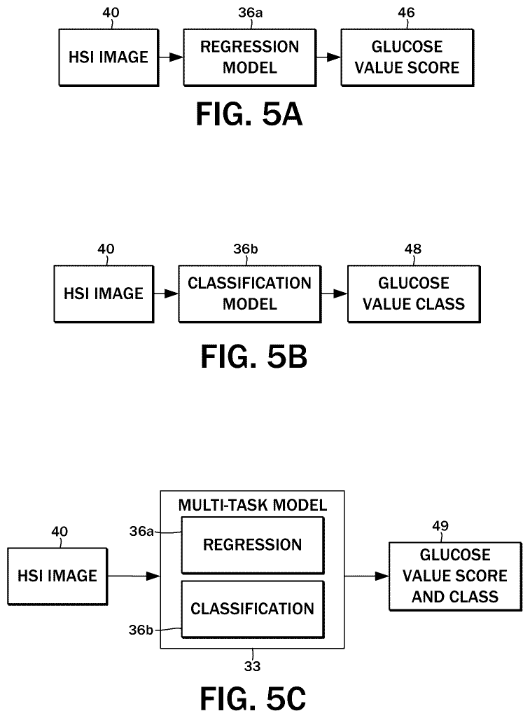

According to an embodiment of the present disclosure illustrated in FIG. 5C, the machine learning model 36 may be implemented as a multi-task learning framework 33 that concurrently handles both regression 36a and classification 36b tasks, generating both the glucose value score and class 49. This approach is intended to harness the shared representations in data to promote improved generalization and performance for each task. The model may be initialized with pre-trained weights, or entirely anew. The pre-trained weights may be obtained from models trained with, for example, the ImageNet image dataset, which is a collection of hierarchically arranged images for visual object recognition. This approach may leverage the advantages of transfer learning, and harness the generic features learned from the vast dataset, and thus lead to faster convergence and improved generalization when fine-tuning on hyperspectral image data. The model may also be trained without any pre-trained data/weights, thus allowing the model to learn features that may be unique to the hyperspectral image dataset without the potential biases from the pre-trained weights.

Again, hyperspectral imaging is understood to capture data across a broad spectrum of wavelengths. This results in a multi-dimensional data set that is rich with information. Various embodiments of the present disclosure contemplate the adaptation of conventional neural network architectures to accommodate this depth of data.

With reference to FIG. 5A, one approach of the machine learning model 36 contemplates a regression model in which the relationship between the inputs and the outputs is a straight line. The hyperspectral image is provided to a regression model 36a, and a glucose score is output. According to one embodiment, the glucose score is the specific blood-glucose concentration value (mg/dL).

FIG. 5B illustrates another approach in which the machine learning model 36 is a classification model. More particularly, the data underlying the training hyperspectral images 40 are divided into different classes of ranges of blood glucose levels. In one implementation, one class of blood glucose levels may be a range from 80-120 mg/dL, another class may be from 120 to 200 mg/dL, and another class may be above 200 mg/dL. Through the training process, the training hyperspectral images 40 may be segregated according to these classes, with an input of the hyperspectral image(s) 40 being provided to the model 36b, and an output of the blood-glucose level class 48 is output. According to one embodiment, this training may be performed by an image classification process, and preferably a convolutional neural network (CNN) or a transformer.

The convolutional neural network may be an EfficientNet, a ResNet, and so forth. In the specific implementation of the machine learning model 36 adapted for evaluating the hyperspectral images, the first convolutional layer that may otherwise be tailored for a three-channel RGB input, may be reconfigured to accommodate the increased channel count. This is so that the machine learning model 36 can effectively capture the intricacies of the multi-spectral image data from the initial analysis steps.

In one implementation, the transformer may be a vision transformer, though any other transformer may be substituted. Conventional transformers, and in particular, vision transformers, the images are understood to be divided into fixed-size patches that are then linearly embedded into vectors. In the example embodiment of the machine learning model 36 adapted for evaluating the hyperspectral images in accordance with the present disclosure, such embedding layer may be modified to handle the greater depth from the additional spectral channels. Additionally, positional encodings may be made congruent with the sequence length of the hyperspectral image patch embeddings. As will be recognized by those having skill in the art, positional encodings may be pivotal for many transformer architectures.

For both paradigms, one adaptation for the hyperspectral images may be to ensure that the initial layers, whether convolutional or embedded, are configured for the profile of hyperspectral data. This allows the richness of the data to be harnessed and provides the foundation for subsequent layers for building hierarchical or sequential abstractions.

The present disclosure also contemplates various embodiments of data augmentation and validation for model training and deployment. To achieve improved levels of robustness and to account for varied spatial contexts during training, random cropping of images may be applied to the hyperspectral image data. This may simulate a larger training data set, and additionally conditions the machine learning model 36 to identify facial features under diverse spatial variations.

The machine learning model 36 may be validated with multiple crops of the hyperspectral image. Specifically, a central crop that target the main facial features of the eyes, nose, and mouth may be used. Furthermore, a series of corner crops from the top left, top right, bottom left, and bottom right may be used to ensure the capture of diverse facial regions. These five crops are applied to a mirrored or horizontal flip image, thereby doubling the number of processed crops. The combination of these structured crops ensure the processing of diverse special subsections of the face, and the predictive capabilities may be improved. Furthermore, averaging the predictions over these ten cropped images is envisioned to result in a more consistent validation metric, and mitigate biases or anomalies from a given facial section.

The machine learning model 36 may be running directly on the smartphone 12 after it has been trained on the training data set 38. Alternatively, the machine learning model 36 may be running remotely on a more powerful computer system such as a server or on a cloud computing platform. In such embodiments, the captured hyperspectral images 40 may be transmitted to the remote system via conventional data transmission modalities implemented on the smartphone 12. Once the input hyperspectral image of the user 14 with unknown blood-glucose levels is provided to the machine learning model 36 via the machine learning interface 34 as discussed above, a result output may be generated thereby. This result output may be transmitted back to the smartphone 12, again via conventional data transmission modalities, and provided to a results processor 50. Depending on the specific type of model implemented (e.g., regression or classification), the output may be either a blood glucose value score, that is, a specific mg/dL value, or a class of ranges of blood glucose values. These outputs may be generated by the user application interface 32.

The machine learning model 36 may be validated based upon a comprehensive set of metrics tailored to general machine learning practices as well as those specific to the application context of blood-glucose level monitoring. Traditional validation metrics associated with classification include accuracy, precision, recall, and F1 score, and those associated with regression models include mean absolute error, mean squared error, and R-squared. Additionally, specialized metrics that elucidate a more nuanced understanding of the machine learning model 36 and its efficacy in predicting glucose levels are contemplated.

In one embodiment, the mean absolute relative difference (MARD) may be utilized for a quantitative assessment of accuracy. MARD is understood to compute the average relative difference between the predicted values and the true reference values. This deviation may be presented as a percentage to reflect the overall accuracy of the machine learning model 36.

MARD is mathematically defined as:

$$MARD = \frac{1}{N} {\sum }_{i = 0}^{N} \vert \frac{( {predicted}_{i} - {reference}_{i} )}{{reference}_{i}} \vert \times 100$$

Where predictedᵢ is the i^{th} predicted value from the machine learning model 36, referenceᵢ is the true i^{th} reference value, and N is the total number of observations. This metric may offer a quantitative assessment of the accuracy of the machine learning model 36 by expressing the average relative difference between the predictions generated thereby and the true reference values as a percentage.

Clark's error grid analysis may be employed to understand the clinical implications of any discrepancies between the prediction and actual values. This analysis is understood to plot predicted values against actual values, and categorizing discrepancies into zones based on their potential therapeutic consequences. Errors within certain zones may have negligible clinical implications, while those in other zones may lead to harmful clinical decisions. Generally, this analysis is contemplated to provide insight into the magnitude of the errors and their potential impact on clinical decision-making, resulting in a more holistic evaluation of the machine-learning model 36 that ensures statistical robustness and practical relevance in real-world medical scenarios.

The use of different cropped sections of an image as contemplated for the validation process discussed above may also be applied to the input hyperspectral image. Each input hyperspectral image may be cropped according to the ten variations discussed above, with the final prediction or output being the average from each of the cropped sections of the hyperspectral image. Thus, comprehensive facial information processing is possible, and the prediction reliability is improved by mitigating the influence of the specific facial region anomalies. The machine learning model 36 can therefore better recognize, process, and predict blood glucose level results.

Although the foregoing examples have considered methods and apparatuses for monitoring blood glucose levels, it will be appreciated that the foregoing features may be adopted to monitor a wide range of health status metrics. For example, the detection of hydration levels from the HSI images may be possible, as well as blood pressure levels and so on. In this regard, the specific references to blood glucose levels herein is for purposes of describing exemplary embodiments of the present disclosure only.

The particulars shown herein are by way of example and for purposes of illustrative discussion of the embodiments of non-invasive and non-contact blood glucose monitoring and are presented in the cause of providing what is believed to be the most useful and readily understood description of the principles and conceptual aspects. In this regard, no attempt is made to show details with more particularity than is necessary, the description taken with the drawings making apparent to those skilled in the art how the several forms of the present disclosure may be embodied in practice.

What is claimed is:

1. A method for deriving a blood glucose level of a user, comprising:
capturing one or more images of the user with a hyperspectral imaging device, the images being defined by a plurality of layered data sets each corresponding to an electromagnetic spectrum band channel;
cropping the one or more images to predefined sets of image excerpts;
feeding the predefined sets of image excerpts to a machine learning model trained on a plurality of correlated pairs of one or more training images associated with training blood glucose measurements; and
generating, with the machine learning model, an estimated blood glucose level for the user corresponding to the one or more images thereof.

2. The method of claim 1, wherein the electromagnetic spectrum band channels of the layered data sets correspond to visible spectrum primary color bands of red, blue, and green.

3. The method of claim 1, wherein one of the electromagnetic spectrum band channels of the layered data sets corresponds to a hyperspectral band channel between approximately 10 nanometers and approximately 0.1 millimeters with 1 nanometer channel steps.

4. The method of claim 1, wherein the one or more images is of a specific body part of the user.

5. The method of claim 4, wherein the body part of the user is selected from a group consisting of: a face, an wrist, and an arm.

6. The method of claim 1, further comprising:
normalizing each of the layered data sets to a constrained minimum and maximum range according to the corresponding electromagnetic spectrum band channel.

7. The method of claim 1, wherein a given one of the predefined sets of image excerpts is selected from a group consisting of: a central crop targeting main facial features, a top-left crop, a top-right crop, a bottom-left crop, a bottom-right crop, a mirrored central crop targeting main facial features, a mirrored top-left crop, a mirrored top-right crop, a mirrored bottom-left crop, and a mirrored bottom-right crop.

8. The method of claim 1, further comprising:
capturing the one or more training images of a plurality of training users with the hyperspectral imaging device, the training images being defined by a plurality of layered data sets each corresponding an electromagnetic spectrum band channel;
capturing the training blood glucose measurements of the training users concurrently with the capturing of the training images; and
feeding one or more correlated pair of the training blood glucose measurement and the training images to the machine learning model.

9. The method of claim 8, further comprising:
training the machine learning model with the correlated pair of the training blood glucose measurement and the training images.

10. The method of claim 1, wherein the machine learning model implements a neural architecture.

11. The method of claim 10, wherein the neural architecture is a convolutional neural network.

12. The method of claim 10, wherein the neural architecture is a vision transformer.

13. The method of claim 8, wherein the convolutional neural network applies a regression model, with the estimated blood glucose level being generated as a numeric score value.

14. The method of claim 8, wherein the convolutional neural network applies a classification model, with the estimated blood glucose level being generated as a class defined by sequential ranges of blood glucose concentrations.

15. The method of claim 8, wherein the convolutional neural network applies a multi-task model including the application of a combination of a regression model and a classification model.

16. An apparatus for monitoring a blood glucose level of a user, the apparatus comprising:
a hyperspectral imaging device, one or more images of the user being captured by the hyperspectral imaging device with each being defined by a plurality of layered data sets each corresponding to an electromagnetic spectrum band channel; and
a glucose level evaluation interface in communication with a machine learning model trained on a plurality of correlated pairs of one or more training images and associated training blood glucose measurements, the one or more images of the user from the hyperspectral imaging device cropped to predefined sets of image excerpts being relayed to the machine learning model, the glucose level evaluation interface being receptive to an estimated blood glucose level generated in response to the one or more images.

17. The apparatus of claim 16, wherein the hyperspectral imaging device and the glucose level evaluation interface are incorporated into a mobile communications device.

18. The apparatus of claim 16, wherein the machine learning model implements a neural architecture.

19. The apparatus of claim 18, wherein the neural architecture is a convolutional neural network.

20. The apparatus of claim 19, wherein the convolutional neural network applies a regression model, with the estimated blood glucose level being generated as a numeric score value.

21. The apparatus of claim 19, wherein the convolutional neural network applies a classification model, with the estimated blood glucose level being generated as a class defined by sequential ranges of blood glucose concentrations.

22. The apparatus of claim 19, wherein the convolutional neural network applies a multi-task model including a combination of a regression model and a classification model.

23. The apparatus of claim 18, wherein the neural architecture is a vision transformer.

24. The apparatus of claim 16, wherein the electromagnetic spectrum band channels of the layered data sets correspond to visible spectrum primary color bands of red, blue, and green.

25. The apparatus of claim 16, wherein one of the electromagnetic spectrum band channels of the layered data sets corresponds to a hyperspectral band channel between approximately 10 nanometers and approximately 0.1 millimeters with 1 nanometer channel steps.

26. The apparatus of claim 16, wherein the one or more images of the user is of a specific body part of the user.

27. The apparatus of claim 26, wherein the body part of the user is selected from a group consisting of: a face, an wrist, and an arm.

28. A non-transitory program storage medium on which are stored instructions executable by a processor or programmable circuit to perform a method for deriving a blood glucose level of a user, the method comprising the steps of:
capturing one or more images of the user with a hyperspectral imaging device, the images being defined by a plurality of layered data sets each corresponding to an electromagnetic spectrum band channel;
cropping the one or more images to predefined sets of image excerpts;
feeding the one or more images of the user to a machine learning model trained on a plurality of correlated pairs of one or more training images associated with training blood glucose measurements; and
generating, with the machine learning model, an estimated blood glucose level for the user corresponding to the one or more images thereof.
